(19) United States
(12) Patent Application Publication
(10) Pub. No.: US 2002/0123784 A1
(43) Pub. Date: Sep. 5, 2002

(54) Implantable medical device having a tri-polar pacing and sensing lead

(76) Inventor: Henk Westendorp, Zutphen (NL)

Correspondence Address: Thomas F. Woods, Medtronic, Inc., 7000 Central Avenue NE, Minneapolis, MN, 55432, US

(21) Appl. No.: 09/750,447

(22) Filed: Dec. 28, 2000

(51) Int. Cl. A61N 1/05

(52) U.S. Cl. 607/122

(57) ABSTRACT

An implantable medical device having a tri-polar pacing and sensing lead is disclosed. The implantable medical device includes a lead capable of insertion into a heart of a patient. The lead further includes a tip electrode, a first ring electrode positioned proximal from the tip electrode, and a second ring electrode positioned proximal from the first ring electrode. A first electrical signal is sensed from the heart via the tip and first ring electrodes. A second electrical signal for pacing the heart is provided to the heart via the tip and second ring electrodes. A digital signal processor is electrically coupled to the tip and first ring electrode. The digital signal processor converts the first set of electrical signals into a digital signal. Pacing electrical circuitry is electrically coupled to the tip and second ring electrodes and coupled to the digital signal processor. The pacing electrical circuitry provides electrical pulses to the heart via the tip and second ring electrodes to pace the heart.

### THE FIELD OF THE INVENTION

The present invention relates generally to implantable medical devices used in conjunction with a heart of a patient. More specifically, the present invention relates to an implantable medical device having a tri-polar pacing and sensing lead for both providing pacing signals to the heart and sensing sensed signals from the heart.

### BACKGROUND OF THE INVENTION

Prior art implantable medical devices, such as cardiac pacemakers, have been devised which closely emulate the electrical activity of the heart. In such devices, sensing both atrial and ventricle depolarization signals and generating pacing signals within both the atrium and the ventricle are possible.

Implantable medical devices need to accurately process sensed signal information of the heart to determine when a genuine cardiac signal has in fact been sensed, and then to accurately identify, or classify, the signal. Often times, artificially created pacing signals interfere with sensed signals. For example, the energy content of the QRS complex occurring during depolarization of the ventricle due to an R-wave signal provided by an implantable medical device is significantly higher than that of the P-wave signal. The R-wave or ventricle pacing spike often appears as a contaminate on the atrial sensing lead. Over sensing of the QRS on the atrial pacemaker lead is common.

Separating cardiac signals from polarization effects and other noise artifacts has always been a substantial problem in this field, and a great deal of effort has been placed on improving implantable medical devices for this purpose. For example, the advent of digital signal processing (DSP) technology has provided a tool, which can be very useful in the environment of an implantable medical device. In DSP technology, the incoming sense signal is converted to a digital signal, e.g., an 8-byte signal at a specified rate. Success of digital signals can be processed with high reliability, in a manner which is essentially hardware-controlled by DSP circuitry. More recently, DSP technology has advanced so as to provide the possibility of a low current chip, which can be used in an implantable medical device to provide significant sense signal processing capability.

Prior art implantable medical devices include multiple leads positioned within various cavities or passageways of a heart of a patient. In one prior art embodiment, each lead contains an electrode. The electrodes are electrically coupled to either sensing or pacing circuitry, or DSP circuitry, in order to provide pacing signals between two electrodes or to receive sensing signals sensed between two electrodes. In addition, some prior art embodiments include one or more leads having two electrodes per lead. These electrode pairs are capable of alternatively supplying a pacing signal to the heart or receiving a sense signal from the heart.

It is understood that a substantial gap or distance should be between two pacing signals to properly pace the heart. Conversely, a relatively small distance or gap should be between two electrodes sensing signals from the heart to minimize pacing artifacts contaminating a sensed signal from the heart.

Several prior art references disclose systems which include both pacing and sensing capabilities within a single lead. These prior art references are listed in Table 1.

TABLE 1

| U.S. Pat. No. | Inventor | Date Issued |
|---|---|---|
| 3,825,015 | Berkovits | 07/23/1974 |
| 4,289,134 | Bernstein | 09/15/1981 |
| 4,355,642 | Alferness | 10/26/1982 |
| 4,848,352 | Pohndorf et al. | 07/18/1989 |
| 5,127,403 | Brownlee | 07/07/1992 |

All patents listed in Table 1 above are hereby incorporated by reference herein in their respective entireties. As those of ordinary skill in the art will appreciate readily upon reading the Summary of the Invention, Detailed Description of the Preferred Embodiments, and claims set forth below, many of the devices and methods disclosed in the patents of Table 1 may be modified advantageously by using the teachings of the present invention. It is clear that none of the prior art references disclose a tri-polar pacing and sensing lead for use with an implantable medical device which optimize both pacing and sensing characteristics.

There is a continuing need for an improved implantable medical device which utilizes a lead having tri-polar pacing and sensing capabilities which maximize the distance between the pacing electrodes and minimizes the distance between the sensing electrodes in order to provide the maximum benefit of both pacing and sensing functions.

### SUMMARY OF THE INVENTION

The present invention overcomes the disadvantages of the prior art by providing a tri-polar pacing and sensing lead capable of providing maximum pacing and sensing capabilities.

The present invention has certain objects. That is, the present invention provides solutions to certain problems existing in the prior art such as: (a) an inability to provide a tri-polar pacing and sensing lead which optimizes both pacing benefits and sensing capabilities; (b) an inability to minimize unwanted pacing artifacts disrupting a sensed signal from the heart; (c) an inability to sense both local and large areas of the atrium and to pace large areas of the atrium with a single lead; (d) an inability to sense local and large areas of the ventricle and to pace large areas of the ventricle with a single lead; (e) an inability to minimize the time required to blank a sensed signal from the heart during a pacing event; (f) an inability to identify gap junction sections of a sensed electrical signal indicating times at which there is minimal heart activity; and (g) an inability to provide a pacing electrical signal to the heart in either the atrium or the ventricle during identified gap junction sections of a sensed signal.

The apparatus and method of the present invention provides certain advantages, including: (a) the ability to provide a tri-polar pacing and sensing lead which optimizes both pacing benefits and sensing capabilities; (b) the ability to minimize unwanted pacing artifacts disrupting a sensed signal from the heart; (c) the ability to sense both local and large areas of the atrium and to pace large areas of the atrium with a single lead; (d) the ability to sense local and large areas of the ventricle and to pace large areas of the ventricle with a single lead; (e) the ability to minimize the time required to blank a sensed signal from the heart during a pacing event; (f) the ability to identify gap junction sections of a sensed electrical signal indicating times at which there is minimal heart activity; and (g) the ability to provide a pacing electrical signal to the heart in either the atrium or the ventricle during identified gap junction sections of a sensed signal.

The apparatus and method of the present invention has certain features, including a tri-polar pacing and sensing lead associated with an implantable medical device. The tri-polar lead includes three electrodes separated from each other to maximize sensing and pacing activities. In particular, the tri-polar lead includes a tip electrode positioned at a distal end of the lead and fabricated from either a porous material or a metal material configured to engage tissue of a heart of a patient. A first ring electrode is located on the tri-polar lead within approximately 1.0 millimeter from the tip electrode. The electrode pair of the tip electrode and the first ring electrode provides local sensing capabilities within either the atrium or the ventricle. A second ring electrode is positioned on the tri-polar lead in the range of approximately 10.0-30.0 millimeters from the tip electrode. The electrode pair of the tip electrode and the second ring electrode provides pacing capabilities of the heart of the patient. In addition, the tip electrode and the second ring electrode may also provide large area sensing capabilities in either the atrium or the ventricle.

Another feature of the present invention is a method of providing pacing electrical pulses to a heart of a patient and of sensing electrical pulses from the heart of the patient. The method includes sensing a sensed electrical signal from the heart between a tip electrode of a lead and a first ring electrode positioned on the lead proximal from the tip electrode. Gap junction sections of the sensed electrical signal are identified which indicate specific times at which there is minimal heart activity. A pacing electrical signal is provided to the heart during the identified gap junction sections of the sensed electrical signal between the tip electrode and a second ring electrode positioned on the lead proximal to the first ring electrode. Another feature of the present invention is the ability to blank the sensed electrical signal corresponding to the time at which the paced electrical signal is provided to the heart of the patient.

### BRIEF DESCRIPTION OF THE DRAWINGS

FIG. 1 is a simplified schematic view of one embodiment of an implantable medical device.

FIG. 2 is a simplified illustration of an implantable medical device with leads positioned within passageways of a heart.

FIG. 3 is a block diagram illustrating the constituent components of an implantable medical device.

FIG. 4 is a simplified schematic view of an implantable medical device with leads positioned within passageways of a heart.

FIG. 5 is a partial block diagram illustrating one embodiment of an implantable medical device used in conjunction with the present invention.

FIG. 6 is a side view illustrating one embodiment of a tri-polar lead in accordance with the present invention.

FIG. 7 is a sectional view of the tri-polar lead shown in FIG. 6.

FIG. 8 is a side view illustrating another embodiment of a tri-polar lead in accordance with the present invention.

FIG. 9 is a sectional view of the tri-polar lead shown in FIG. 8.

FIG. 10 is a schematic diagram illustrating one embodiment of an implantable medical device in accordance with the present invention.

FIG. 11 is a schematic diagram illustrating another embodiment of an implantable medical device in accordance with the present invention.

FIGS. 12-14 are various graphs illustrating an electrical signal sensed from the heart of a patient via a tri-polar lead in accordance with the present invention.

FIG. 15 is a flow chart illustrating various steps in the pacing and sensing of a heart of a patient in accordance with the present invention.

FIG. 16 is a flow chart illustrating various steps in the pacing and sensing of a heart of a patient in accordance with the present invention.

### DETAILED DESCRIPTION OF THE PREFERRED EMBODIMENTS

In the following detailed description of the preferred embodiments, reference is made to the accompanying drawings which form a part hereof, and in which is shown by way of illustration specific embodiments in which the invention may be practiced. It is to be understood that other embodiments may be utilized and structural or logical changes may be made without departing from the scope of the present invention. The following detailed description, therefore, is not to be taken in a limiting sense, and the scope of the present invention is defined by the appended claims.

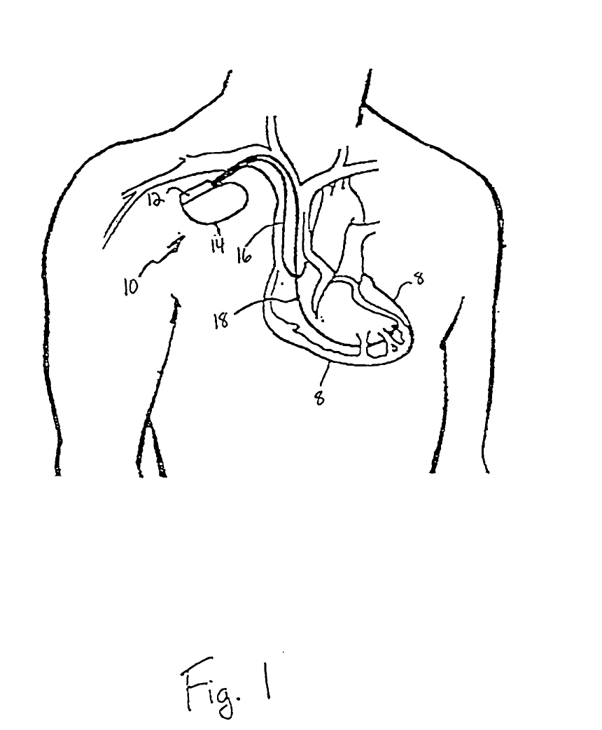

FIG. 1 is a simplified schematic view of one embodiment of implantable medical device (“IMD”) 10 of the present invention. IMD 10 shown in FIG. 1 is a pacemaker comprising at least one of pacing and sensing leads 16 and 18 attached to hermetically sealed enclosure 14 and implanted near human or mammalian heart 8. Pacing and sensing leads 16 and 18 sense electrical signals attendant to the depolarization and re-polarization of the heart 8, and further provide pacing pulses for causing depolarization of cardiac tissue in the vicinity of the distal ends thereof. Leads 16 and 18 may have uni-polar or bipolar electrodes disposed thereon, as is well known in the art. Examples of IMD 10 include implantable cardiac pacemakers disclosed in U.S. Pat. No. 5,158,078 to Bennett et al., U.S. Pat. No. 5,312,453 to Shelton et al. or U.S. Pat. No. 5,144,949 to Olson, all hereby incorporated by reference herein, each in its respective entirety.

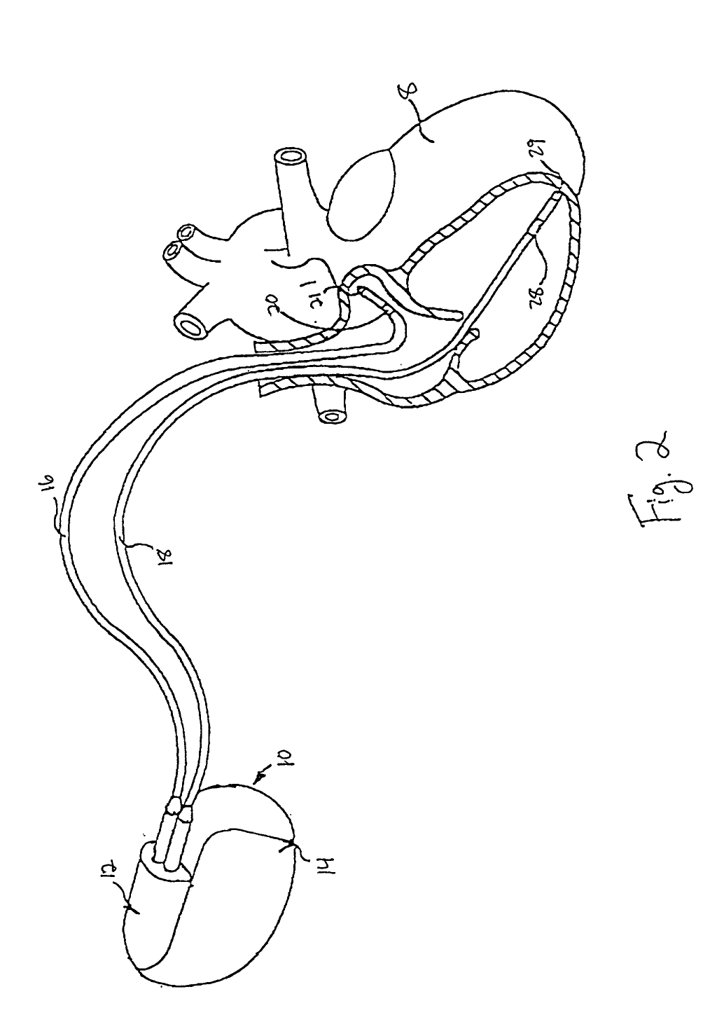

FIG. 2 shows connector module 12 and hermetically sealed enclosure 14 of IMD 10 located in and near human or mammalian heart 8. Atrial and ventricular pacing leads 16 and 18 extend from connector header module 12 to the right atrium and ventricle, respectively, of heart 8. Atrial electrodes 20 and 21 disposed at the distal end of atrial pacing lead 16 are located in the right atrium. Ventricular electrodes 28 and 29 at the distal end of ventricular pacing lead 18 are located in the right ventricle.

FIG. 3 shows a block diagram illustrating the constituent components of IMD 10 in accordance with one embodiment of the present invention, where IMD 10 is pacemaker having a microprocessor-based architecture. IMD 10 is shown as including activity sensor or accelerometer 11, which is preferably a piezoceramic accelerometer bonded to a hybrid circuit located inside enclosure 14. Activity sensor 11 typically (although not necessarily) provides a sensor output that varies as a function of a measured parameter relating to a patient's metabolic requirements. For the sake of convenience, IMD 10 in FIG. 3 is shown with lead 18 only connected thereto; similar circuitry and connections not explicitly shown in FIG. 3 apply to lead 16.

IMD 10 in FIG. 3 is most preferably programmable by means of an external programming unit (not shown in the Figures). One such programmer is the commercially available Medtronic Model 9790 programmer, which is microprocessor-based and provides a series of encoded signals to IMD 10, typically through a programming head which transmits or telemeters radio-frequency (RF) encoded signals to IMD 10. Such a telemetry system is described in U.S. Pat. No. 5,312,453 to Wyborny et al., hereby incorporated by reference herein in its entirety. The programming methodology disclosed in Wyborny et al.'s '453 patent is identified herein for illustrative purposes only. Any of a number of suitable programming and telemetry methodologies known in the art may be employed so long as the desired information is transmitted to and from the pacemaker.

As shown in FIG. 3, lead 18 is coupled to node 50 in IMD 10 through input capacitor 52. Activity sensor or accelerometer 11 is most preferably attached to a hybrid circuit located inside hermetically sealed enclosure 14 of IMD 10. The output signal provided by activity sensor 11 is coupled to input/output circuit 54. Input/output circuit 54 contains analog circuits for interfacing to heart 8, activity sensor 11, antenna 56 and circuits for the application of stimulating pulses to heart 8. The rate of heart 8 is controlled by software-implemented algorithms stored microcomputer circuit 58.

Microcomputer circuit 58 preferably comprises on-board circuit 60 and off-board circuit 62. Circuit 58 may correspond to a microcomputer circuit disclosed in U.S. Pat. No. 5,312,453 to Shelton et al., hereby incorporated by reference herein in its entirety. On-board circuit 60 preferably includes microprocessor 64, system clock circuit 66 and on-board RAM 68 and ROM 70. Off-board circuit 62 preferably comprises a RAM/ROM unit. On-board circuit 60 and off-board circuit 62 are each coupled by data communication bus 72 to digital controller/timer circuit 74. Microcomputer circuit 58 may comprise a custom integrated circuit device augmented by standard RAM/ROM components.

Electrical components shown in FIG. 3 are powered by an appropriate implantable battery power source 76 in accordance with common practice in the art. For the sake of clarity, the coupling of battery power to the various components of IMD 10 is not shown in the Figures. Antenna 56 is connected to input/output circuit 54 to permit uplink/downlink telemetry through RF transmitter and receiver telemetry unit 78. By way of example, telemetry unit 78 may correspond to that disclosed in U.S. Pat. No. 4,566,063 issued to Thompson et al., hereby incorporated by reference herein in its entirety, or to that disclosed in the above-referenced '453 patent to Wyborny et al. It is generally preferred that the particular programming and telemetry scheme selected permit the entry and storage of cardiac rate-response parameters. The specific embodiments of antenna 56, input/output circuit 54 and telemetry unit 78 presented herein are shown for illustrative purposes only, and are not intended to limit the scope of the present invention.

Continuing to refer to FIG. 3, VREF and Bias circuit 82 most preferably generates stable voltage reference and bias currents for analog circuits included in input/output circuit 54. Analog-to-digital converter (ADC) and multi-plexer unit 84 digitizes analog signals and voltages to provide “real-time” telemetry intracardiac signals and battery end-of-life (EOL) replacement functions. Operating commands for controlling the timing of IMD 10 are coupled by data bus 72 to digital controller/timer circuit 74, where digital timers and counters establish the overall escape interval of the IMD 10 as well as various refractory, blanking and other timing windows for controlling the operation of peripheral components disposed within input/output circuit 54.

Digital controller/timer circuit 74 is preferably coupled to sensing circuitry, including sense amplifier 88, peak sense and threshold measurement unit 90 and comparator/threshold detector 92. Circuit 74 is further preferably coupled to electrogram (EGM) amplifier 94 for receiving amplified and processed signals sensed by lead 18. Sense amplifier 88 amplifies sensed electrical cardiac signals and provides an amplified signal to peak sense and threshold measurement circuitry 90, which in turn provides an indication of peak sensed voltages and measured sense amplifier threshold voltages on multiple conductor signal path 67 to digital controller/timer circuit 74. An amplified sense amplifier signal is then provided to comparator/threshold detector 92. By way of example, sense amplifier 88 may correspond to that disclosed in U.S. Pat. No. 4,379,459 to Stein, hereby incorporated by reference herein in its entirety.

The electrogram signal provided by EGM amplifier 94 is employed when IMD 10 is being interrogated by an external programmer to transmit a representation of a cardiac analog electrogram. See, for example, U.S. Pat. No. 4,556,063 to Thompson et al., hereby incorporated by reference herein in its entirety. Output pulse generator 96 provides pacing stimuli to patient's heart 8 through coupling capacitor 98 in response to a pacing trigger signal provided by digital controller/timer circuit 74 each time the escape interval times out, an externally transmitted pacing command is received or in response to other stored commands as is well known in the pacing art. By way of example, output amplifier 96 may correspond generally to an output amplifier disclosed in U.S. Pat. No. 4,476,868 to Thompson, hereby incorporated by reference herein in its entirety.

The specific embodiments of input amplifier 88, output amplifier 96 and EGM amplifier 94 identified herein are presented for illustrative purposes only, and are not intended to be limiting in respect of the scope of the present invention. The specific embodiments of such circuits may not be critical to practicing some embodiments of the present invention so long as they provide means for generating a stimulating pulse and are capable of providing signals indicative of natural or stimulated contractions of heart 8.

In some preferred embodiments of the present invention, IMD 10 may operate in various non-rate-responsive modes, including, but not limited to, DDD, DDI, VVI, VOO and VVT modes. In other preferred embodiments of the present invention, IMD 10 may operate in various rate-responsive, including, but not limited to, DDDR, DDIR, VVIR, VOOR and VVTR modes. Some embodiments of the present invention are capable of operating in both non-rate-responsive and rate responsive modes. Moreover, in various embodiments of the present invention IMD 10 may be programmably configured to operate so that it varies the rate at which it delivers stimulating pulses to heart 8 only in response to one or more selected sensor outputs being generated. Numerous pacemaker features and functions not explicitly mentioned herein may be incorporated into IMD 10 while remaining within the scope of the present invention.

The present invention is not limited in scope to single-sensor or dual-sensor pacemakers, and is not limited to IMD's comprising activity or pressure sensors only. Nor is the present invention limited in scope to single-chamber pacemakers, single-chamber leads for pacemakers or single-sensor or dual-sensor leads for pacemakers. Thus, various embodiments of the present invention may be practiced in conjunction with more than two leads or with multiple-chamber pacemakers, for example. At least some embodiments of the present invention may be applied equally well in the contexts of single-, dual-, triple- or quadruple- chamber pacemakers or other types of IMD's. See, for example, U.S. Pat. No. 5,800,465 to Thompson et al., hereby incorporated by reference herein in its entirety, as are all U.S. Patents referenced therein.

IMD 10 may also be a pacemaker-cardioverter- defibrillator (“PCD”) corresponding to any of numerous commercially available implantable PCD's. Various embodiments of the present invention may be practiced in conjunction with PCD's such as those disclosed in U.S. Pat. No. 5,545,186 to Olson et al., U.S. Pat. No. 5,354,316 to Keimel, U.S. Pat. No. 5,314,430 to Bardy, U.S. Pat. No. 5,131,388 to Pless and U.S. Pat. No. 4,821,723 to Baker et al., all hereby incorporated by reference herein, each in its respective entirety.

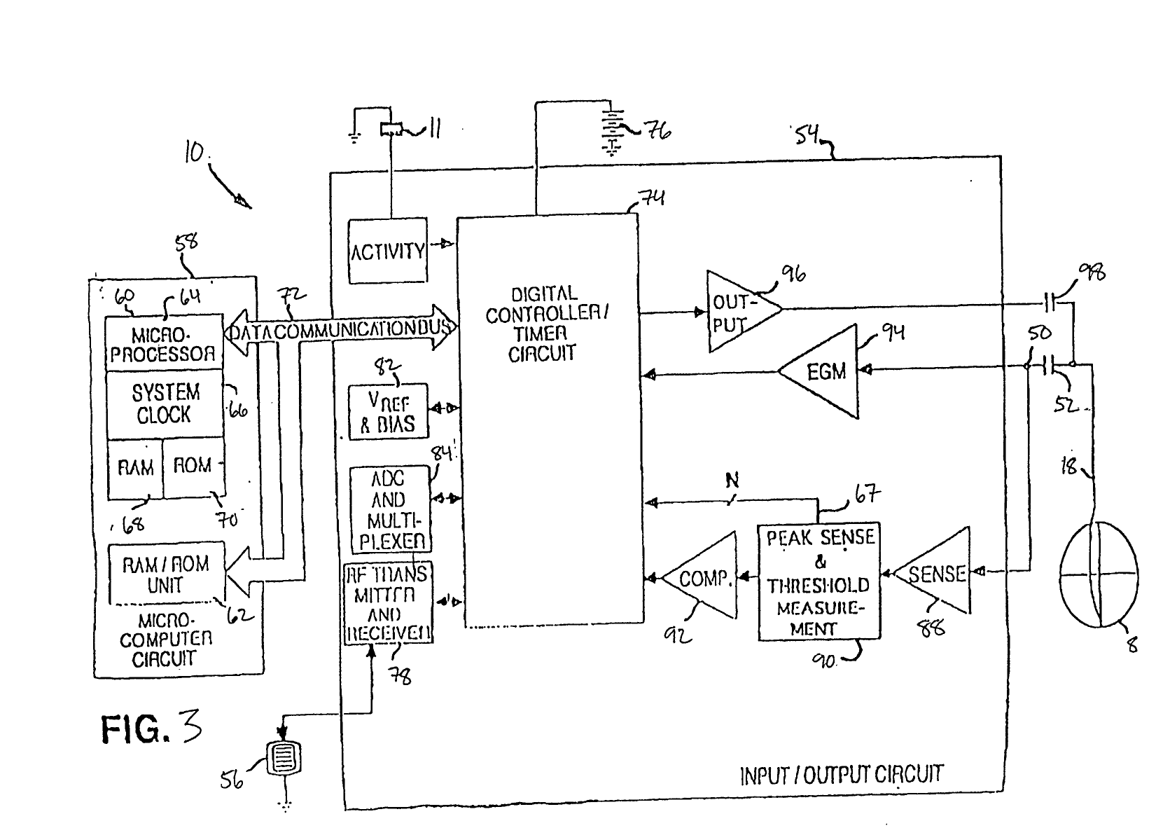

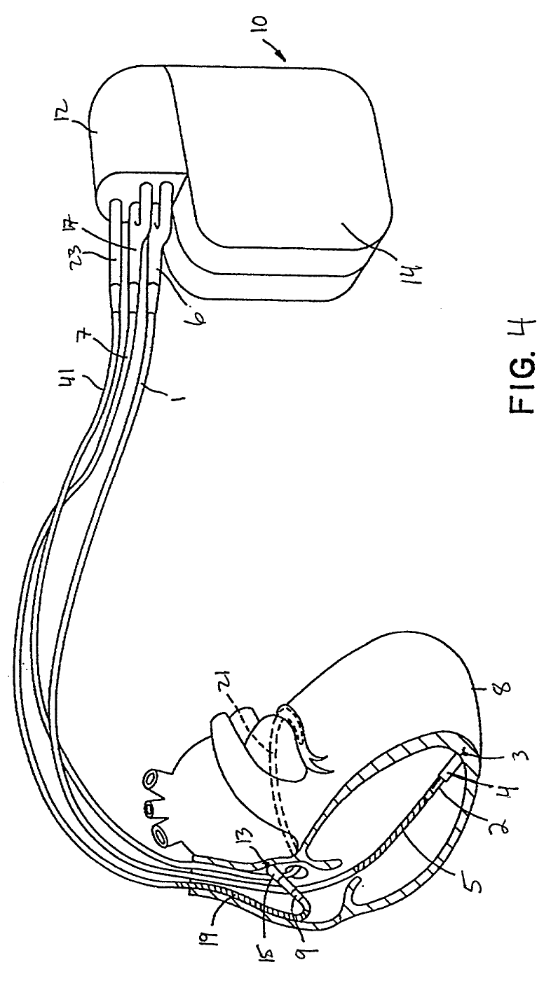

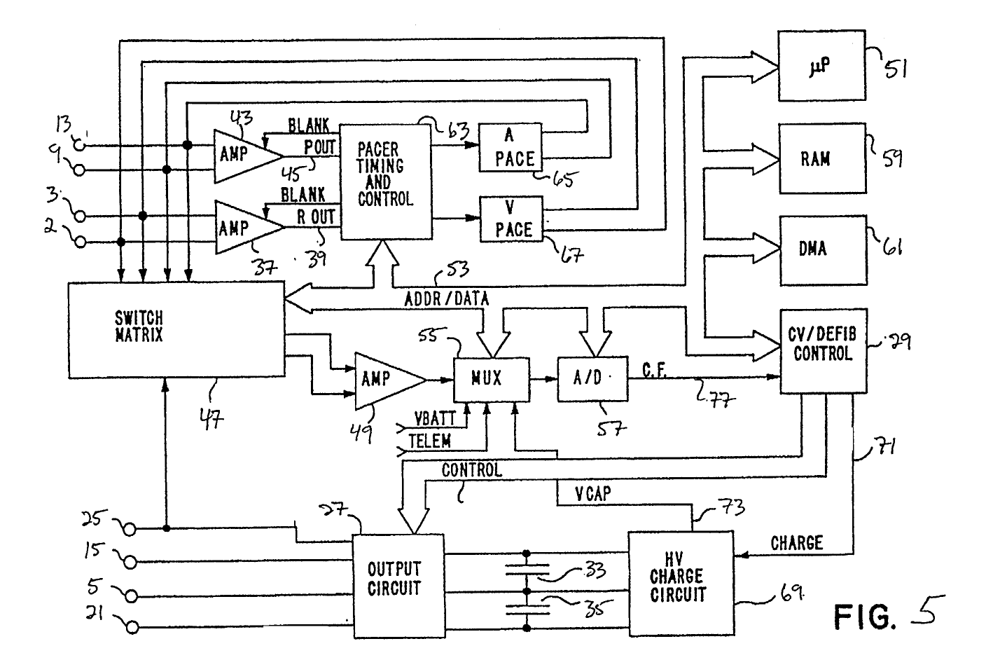

FIGS. 4 and 5 illustrate one embodiment of IMD 10 and a corresponding lead set of the present invention, where IMD 10 is a PCD. In FIG. 4, the ventricular lead takes the form of leads disclosed in U.S. Pat. Nos. 5,099,838 and 5,314,430 to Bardy, and includes an elongated insulative lead body 1 carrying three concentric coiled conductors separated from one another by tubular insulative sheaths. Located adjacent the distal end of lead 1 are ring electrode 2, extendable helix electrode 3 mounted retractably within insulative electrode head 4 and elongated coil electrode 5. Each of the electrodes is coupled to one of the coiled conductors within lead body 1. Electrodes 2 and 3 are employed for cardiac pacing and for sensing ventricular depolarizations. At the proximal end of the lead is bifurcated connector 6 which carries three electrical connectors, each coupled to one of the coiled conductors. Defibrillation electrode 5 may be fabricated from platinum, platinum alloy or other materials known to be usable in implantable defibrillation electrodes and may be about 5 cm in length.

The atrial/SVC lead shown in FIG. 4 includes elongated insulative lead body 7 carrying three concentric coiled conductors separated from one another by tubular insulative sheaths corresponding to the structure of the ventricular lead. Located adjacent the J-shaped distal end of the lead are ring electrode 9 and extendable helix electrode 13 mounted retractably within an insulative electrode head 15. Each of the electrodes is coupled to one of the coiled conductors within lead body 7. Electrodes 13 and 9 are employed for atrial pacing and for sensing atrial depolarizations. Elongated coil electrode 19 is provided proximal to electrode 9 and coupled to the third conductor within lead body 7. Electrode 19 preferably is 10 cm in length or greater and is configured to extend from the SVC toward the tricuspid valve. In one embodiment of the present invention, approximately 5 cm of the right atrium/SVC electrode is located in the right atrium with the remaining 5 cm located in the SVC. At the proximal end of the lead is bifurcated connector 17 carrying three electrical connectors, each coupled to one of the coiled conductors.

The coronary sinus lead shown in FIG. 4 assumes the form of a coronary sinus lead disclosed in the above cited '838 patent issued to Bardy, and includes elongated insulative lead body 41 carrying one coiled conductor coupled to an elongated coiled defibrillation electrode 21. Electrode 21, illustrated in broken outline in FIG. 4, is located within the coronary sinus and great vein of the heart. At the proximal end of the lead is connector plug 23 carrying an electrical connector coupled to the coiled conductor. The coronary sinus/great vein electrode 41 may be about 5 cm in length.

Implantable PCD 10 is shown in FIG. 4 in combination with leads 1, 7 and 41, and lead connector assemblies 23, 17 and 6 inserted into connector block 12. Optionally, insulation of the outward facing portion of housing 14 of PCD 10 may be provided using a plastic coating such as parylene or silicone rubber, as is employed in some uni-polar cardiac pacemakers. The outward facing portion, however, may be left uninsulated or some other division between insulated and uninsulated portions may be employed. The uninsulated portion of housing 14 serves as a subcutaneous defibrillation electrode to defibrillate either the atria or ventricles. Lead configurations other that those shown in FIG. 4 may be practiced in conjunction with the present invention, such as those shown in U.S. Pat. No. 5,690,686 to Min et al., hereby incorporated by reference herein in its entirety.

FIG. 5 is a functional schematic diagram of one embodiment of implantable PCD 10 of the present invention. This diagram should be taken as exemplary of the type of device in which various embodiments of the present invention may be embodied, and not as limiting, as it is believed that the invention may be practiced in a wide variety of device implementations, including cardioverter and defibrillators which do not provide anti-tachycardia pacing therapies.

IMD 10 is provided with an electrode system. If the electrode configuration of FIG. 4 is employed, the correspondence to the illustrated electrodes is as follows. Electrode 25 in FIG. 5 includes the uninsulated portion of the housing of PCD 10. Electrodes 25, 15, 21 and 5 are coupled to high voltage output circuit 27, which includes high voltage switches controlled by CV/defib control logic 29 via control bus 31. Switches disposed within circuit 27 determine which electrodes are employed and which electrodes are coupled to the positive and negative terminals of the capacitor bank (which includes capacitors 33 and 35) during delivery of defibrillation pulses.

Electrodes 2 and 3 are located on or in the ventricle and are coupled to the R-wave amplifier 37, which preferably takes the form of an automatic gain controlled amplifier providing an adjustable sensing threshold as a function of the measured R-wave amplitude. A signal is generated on R-out line 39 whenever the signal sensed between electrodes 2 and 3 exceeds the present sensing threshold.

Electrodes 9 and 13 are located on or in the atrium and are coupled to the P-wave amplifier 43, which preferably also takes the form of an automatic gain controlled amplifier providing an adjustable sensing threshold as a function of the measured P-wave amplitude. A signal is generated on P-out line 45 whenever the signal sensed between electrodes 9 and 13 exceeds the present sensing threshold. The general operation of R-wave and P-wave amplifiers 37 and 43 may correspond to that disclosed in U.S. Pat. No. 5,117,824, by Keimel et al., issued Jun. 2, 1992, for “An Apparatus for Monitoring Electrical Physiologic Signals”, hereby incorporated by reference herein in its entirety.

Switch matrix 47 is used to select which of the available electrodes are coupled to wide band (0.5-200 Hz) amplifier 49 for use in digital signal analysis. Selection of electrodes is controlled by the microprocessor 51 via data/address bus 53, which selections may be varied as desired. Signals from the electrodes selected for coupling to bandpass amplifier 49 are provided to multi-plexer 55, and thereafter converted to multi-bit digital signals by A/D converter 57, for storage in random access memory 59 under control of direct memory access circuit 61. Microprocessor 51 may employ digital signal analysis techniques to characterize the digitized signals stored in random access memory 59 to recognize and classify the patient's heart rhythm employing any of the numerous signal processing methodologies known to the art.

The remainder of the circuitry is dedicated to the provision of cardiac pacing, cardioversion and defibrillation therapies, and, for purposes of the present invention may correspond to circuitry known to those skilled in the art. The following exemplary apparatus is disclosed for accomplishing pacing, cardioversion and defibrillation functions. Pacer timing/control circuitry 63 preferably includes programmable digital counters which control the basic time intervals associated with DDD, VVI, DVI, VDD, AAI, DDI and other modes of single and dual chamber pacing well known to the art. Circuitry 63 also preferably controls escape intervals associated with anti-tachyarrhythmia pacing in both the atrium and the ventricle, employing any anti-tachyarrhythmia pacing therapies known to the art.

Intervals defined by pacing circuitry 63 include atrial and ventricular pacing escape intervals, the refractory periods during which sensed P-waves and R-waves are ineffective to restart timing of the escape intervals and the pulse widths of the pacing pulses. The durations of these intervals are determined by microprocessor 51, in response to stored data in memory 59 and are communicated to pacing circuitry 63 via address/data bus 53. Pacer circuitry 63 also determines the amplitude of the cardiac pacing pulses under control of microprocessor 51.

During pacing, escape interval counters within pacer timing/control circuitry 63 are reset upon sensing of R-waves and P-waves as indicated by a signals on lines 39 and 45, and in accordance with the selected mode of pacing on time-out trigger generation of pacing pulses by pacer output circuitry 65 and 67, which are coupled to electrodes 9, 13, 2 and 3. Escape interval counters are also reset on generation of pacing pulses and thereby control the basic timing of cardiac pacing functions, including anti-tachyarrhythmia pacing. The durations of the intervals defined by escape interval timers are determined by microprocessor 51 via data/address bus 53. The value of the count present in the escape interval counters when reset by sensed R-waves and P-waves may be used to measure the durations of R-R intervals, P-P intervals, P-R intervals and R-P intervals, which measurements are stored in memory 59 and used to detect the presence of tachyarrhythmias.

Microprocessor 51 most preferably operates as an interrupt driven device, and is responsive to interrupts from pacer timing/control circuitry 63 corresponding to the occurrence sensed P-waves and R-waves and corresponding to the generation of cardiac pacing pulses. Those interrupts are provided via data/address bus 53. Any necessary mathematical calculations to be performed by microprocessor 51 and any updating of the values or intervals controlled by pacer timing/control circuitry 63 take place following such interrupts.

Detection of atrial or ventricular tachyarrhythmias, as employed in the present invention, may correspond to tachyarrhythmia detection algorithms known in the art. For example, the presence of an atrial or ventricular tachyarrhythmia may be confirmed by detecting a sustained series of short R-R or P-P intervals of an average rate indicative of tachyarrhythmia or an unbroken series of short R-R or P-P intervals. The suddenness of onset of the detected high rates, the stability of the high rates, and a number of other factors known in the art may also be measured at this time. Appropriate ventricular tachyarrhythmia detection methodologies measuring such factors are described in U.S. Pat. No. 4,726,380 issued to Vollmann, U.S. Pat. No. 4,880,005 issued to Pless et al. and U.S. Pat. No. 4,830,006 issued to Haluska et al., all incorporated by reference herein, each in its respective entirety. An additional set of tachycardia recognition methodologies is disclosed in the article “Onset and Stability for Ventricular Tachyarrhythmia Detection in an Implantable Pacer-Cardioverter-Defibrillator” by Olson et al., published in Computers in Cardiology, Oct. 7-10, 1986, IEEE Computer Society Press, pages 167-170, also incorporated by reference herein in its entirety. Atrial fibrillation detection methodologies are disclosed in Published PCT Application Ser. No. US92/02829, Publication No. WO92/18198, by Adams et al., and in the article “Automatic Tachycardia Recognition”, by Arzbaecher et al., published in PACE, May-June, 1984, pp. 541-547, both of which are incorporated by reference herein in their entireties.

In the event an atrial or ventricular tachyarrhythmia is detected and an anti-tachyarrhythmia pacing regimen is desired, appropriate timing intervals for controlling generation of anti-tachyarrhythmia pacing therapies are loaded from microprocessor 51 into the pacer timing and control circuitry 63, to control the operation of the escape interval counters therein and to define refractory periods during which detection of R-waves and P-waves is ineffective to restart the escape interval counters.

Alternatively, circuitry for controlling the timing and generation of anti-tachycardia pacing pulses as described in U.S. Pat. No. 4,577,633, issued to Berkovits et al. on Mar. 25, 1986, U.S. Pat. No. 4,880,005, issued to Pless et al. on Nov. 14, 1989, U.S. Pat. No. 4,726,380, issued to Vollmann et al. on Feb. 23, 1988 and U.S. Pat. No. 4,587,970, issued to Holley et al. on May 13, 1986, all of which are incorporated herein by reference in their entireties, may also be employed.

In the event that generation of a cardioversion or defibrillation pulse is required, microprocessor 51 may employ an escape interval counter to control timing of such cardioversion and defibrillation pulses, as well as associated refractory periods. In response to the detection of atrial or ventricular fibrillation or tachyarrhythmia requiring a cardioversion pulse, microprocessor 51 activates cardioversion/defibrillation control circuitry 29, which initiates charging of the high voltage capacitors 33 and 35 via charging circuit 69, under the control of high voltage charging control line 71. The voltage on the high voltage capacitors is monitored via VCAP line 73, which is passed through multi-plexer 55 and in response to reaching a predetermined value set by microprocessor 51, results in generation of a logic signal on Cap Full (CF) line 77 to terminate charging. Thereafter, timing of the delivery of the defibrillation or cardioversion pulse is controlled by pacer timing/control circuitry 63. Following delivery of the fibrillation or tachycardia therapy microprocessor 51 returns the device to q cardiac pacing mode and awaits the next successive interrupt due to pacing or the occurrence of a sensed atrial or ventricular depolarization.

Several embodiments of appropriate systems for the delivery and synchronization of ventricular cardioversion and defibrillation pulses and for controlling the timing functions related to them are disclosed in U.S. Pat. No. 5,188,105 to Keimel, U.S. Pat. No. 5,269,298 to Adams et al. and U.S. Pat. No. 4,316,472 to Mirowski et al., hereby incorporated by reference herein, each in its respective entirety. Any known cardioversion or defibrillation pulse control circuitry is believed to be usable in conjunction with various embodiments of the present invention, however. For example, circuitry controlling the timing and generation of cardioversion and defibrillation pulses such as that disclosed in U.S. Pat. No. 4,384,585 to Zipes, U.S. Pat. No. 4,949,719 to Pless et al., or U.S. Pat. No. 4,375,817 to Engle et al., all hereby incorporated by reference herein in their entireties, may also be employed.

Continuing to refer to FIG. 5, delivery of cardioversion or defibrillation pulses is accomplished by output circuit 27 under the control of control circuitry 29 via control bus 31. Output circuit 27 determines whether a monophasic or biphasic pulse is delivered, the polarity of the electrodes and which electrodes are involved in delivery of the pulse. Output circuit 27 also includes high voltage switches which control whether electrodes are coupled together during delivery of the pulse. Alternatively, electrodes intended to be coupled together during the pulse may simply be permanently coupled to one another, either exterior to or interior of the device housing, and polarity may similarly be pre-set, as in current implantable defibrillators. An example of output circuitry for delivery of biphasic pulse regimens to multiple electrode systems may be found in the above cited patent issued to Mehra and in U.S. Pat. No. 4,727,877, hereby incorporated by reference herein in its entirety.

An example of circuitry which may be used to control delivery of monophasic pulses is disclosed in U.S. Pat. No. 5,163,427 to Keimel, also incorporated by reference herein in its entirety. Output control circuitry similar to that disclosed in U.S. Pat. No. 4,953,551 to Mehra et al. or U.S. Pat. No. 4,800,883 to Winstrom, both incorporated by reference herein in their entireties, may also be used in conjunction with various embodiments of the present invention to deliver biphasic pulses.

Alternatively, IMD 10 may be an implantable nerve stimulator or muscle stimulator such as that disclosed in U.S. Pat. No. 5,199,428 to Obel et al., U.S. Pat. No. 5,207,218 to Carpentier et al. or U.S. Pat. No. 5,330,507 to Schwartz, or an implantable monitoring device such as that disclosed in U.S. Pat. No. 5,331,966 issued to Bennet et al., all of which are hereby incorporated by reference herein, each in its respective entirety. The present invention is believed to find wide application to any form of implantable electrical device for use in conjunction with electrical leads.

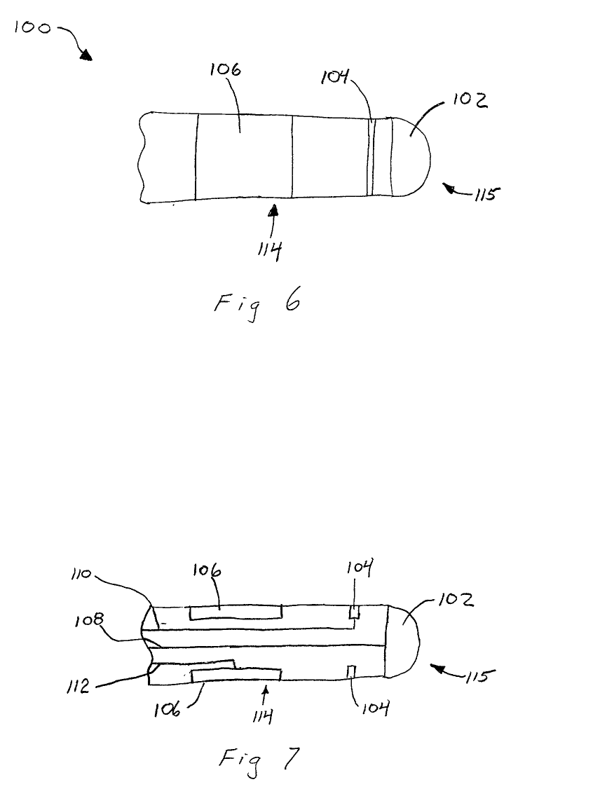

FIG. 6 and 7 are a side view and a sectional view, respectfully, of tri-polar pacing and sensing lead 100, respectively. Lead 100 includes electrodes 102, 104, and 106, with associated conductors 108, 110, and 112, and body portion 114. Lead 100 may be substituted for pacing and sensing leads 16 and 18 shown and described with reference to FIGS. 1-3 and for leads 1 and 7 shown and described with reference to FIG. 4. Thus, it is understood that tri-polar pacing and sensing lead 100 may be inserted into either the atrium or ventricle and may properly pace and sense as described herein in either the atrium or ventricle.

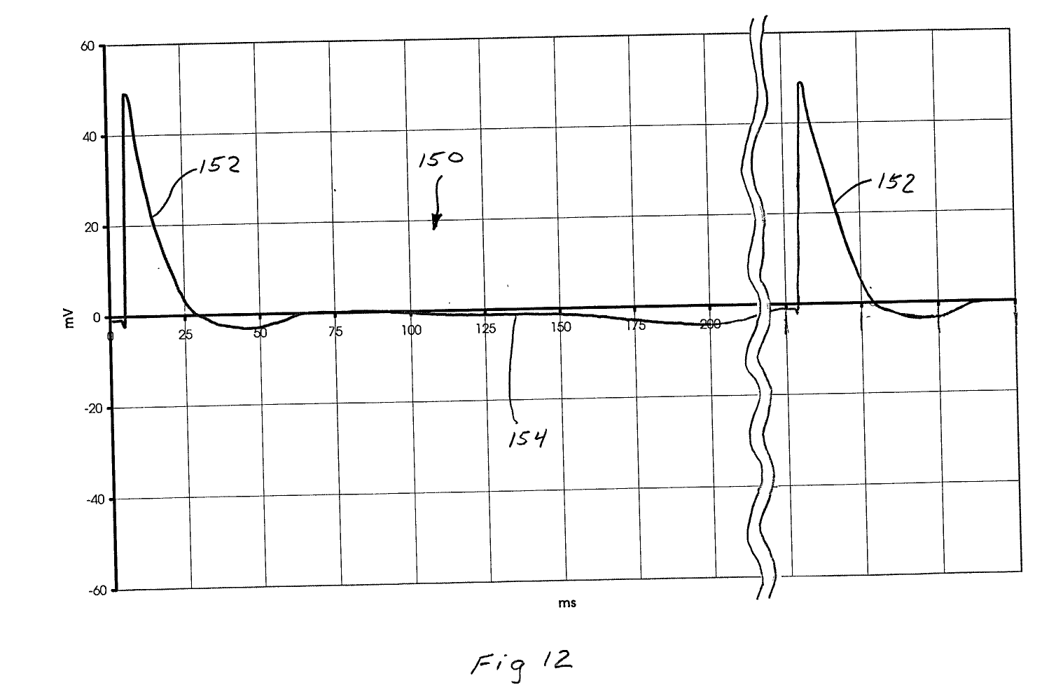

In one preferred embodiment, electrode 102 is considered a tip electrode and is located at proximal end 115 of lead 100. Electrode 102 is formed from either a porous material or from any other conducting material, such as platinum. As shown in FIGS. 6 and 7, electrode 102 may be fabricated in a bulb-like configuration to maximize contact with heart tissue. Electrodes 104 and 106 are often referred to as ring electrodes and are formed from any conducting material, such as platinum. Conductors 108, 110, and 112 represent standard electrical conductors known in the art and electrically couple electrodes 102, 104, and 106, respectively to electrical circuitry within IMD 10, as will be shown and described with reference to FIGS. 10 and 11.

Tip electrode 102 is an electrically common electrode, common to both electrodes 104 and 106. Thus, electrodes 102 and 104 form a first electrode pair, while electrodes 102 and 106 form a second electrode pair. In one embodiment, ring electrode 104 is located on body portion 114 of lead 102 in the range of approximately less than 3.0 millimeters, and preferably in the range of approximately 1.0-3.0 millimeters from tip electrode 102. In one embodiment, ring electrode 106 is located on body portion 114 of lead 102 in the range of approximately 10.0-30.0 millimeters from tip electrode 102. In addition, in one embodiment, ring electrode 104 has a width in the range of approximately 3.0-10.0 millimeters, while ring electrode 106 has a width in the range of approximately 3.0-10.0 millimeters. In one embodiment, ring electrode 106 may have either a larger or smaller width than ring electrode 104. However, in another embodiment, ring electrodes 104 and 106 may be approximately equal in length. It is understood that the illustrations within the figures may not be proportionally accurate, and the scaling of the figures should not be construed as a limitation to the attached claims.

IMD 10 needs to accurately process sensed signal information of the heart to determine when a genuine cardiac signal has in fact been sensed, and then to accurately identify, or classify, the signal. Often times, artificially created pacing signals interfere with sensed signals. For example, the energy content of the QRS complex occurring during depolarization of the ventricle due to an R-wave signal provided by an IMD is significantly higher than that of the P-wave signal. The R-wave or ventricle pacing spike often appears as a contaminate on the atrial sensing lead. Over sensing of the QRS on the atrial pacemaker lead is common.

Prior art IMDs, which pace and sense via two electrodes positioned on a single electrode, are incapable of optimizing both pacing and sensing functions since the pacing function requires a relatively substantial distance between electrodes and the sensing function requires a relatively small distance between electrodes. Further, it is often advantageous to sense within either or both of the atrium or ventricle. A strong signal or impression of a particular disease of heart 8 or of specific muscle of the heart can be sensed via an electrode positioned within the ventricle. Conversely, a strong signal or impression of heart conditions, such as a tachycardia or arrhythmic condition, can be sensed via an electrode positioned within the atrium.

In the embodiments shown in FIGS. 6 and 7, electrodes 102 and 104 represent a first electrode pair capable of sensing a local electrical signal representative of functions of the heart in the vicinity of lead 100. More in particular, electrode pair 102 and 104 is capable of sensing electrical signals or pulses which include information relating to muscle tissue, a heart abnormality, a tachycardia condition, an arrhythmic condition, or blood flow within the heart, for example. Electrode pair 102 and 104 is also capable of sensing electrical signals generated from outside sources, such as electrical signals generated from a pacing activity of IMD 10.

Electrode pair 102 and 106 is capable of providing a pacing signal, or a series of current pulses or blasts, to the heart of the patient in general, and to tissue immediately surrounding lead 100, in particular. Each current pulse causes the heart, or specific cardiac tissue of the heart, to contract, thereby causing depolarization of the heart, as previously discussed. In addition, electrode pair 102 and 106 is capable of providing sensing functions of heart 8, as previously discussed with reference to electrode pair 102 and 104.

Tri-polar pacing and sensing lead 100 differs from prior art leads in that lead 100 includes three strategically spaced electrodes (102, 104, 106), rather than only one or two leads. In some prior art designs, two electrodes, such as electrodes 102 and 106 provide both pacing and sensing functions at altering time intervals. In other prior art designs, each lead includes a single electrode. Pacing and sensing functions are provided between two electrodes on separate leads. With the present invention, electrode 104 has been incorporated into lead 100, thereby permitting precise sensing of local atrial or ventricle events and conditions, while minimizing the unwanted effects of noise, such as due to pacing events. It is understood by those in art that in order to pace tissues of the heart, it is desirable to provide relatively large spacing between the positive and negative electrodes, such as greater than 10.0 millimeters, and preferably in the range of approximately 10.0-30.0 millimeters. Conversely, it is important to minimize the spacing between the positive and negative electrodes performing the sensing functions, such as less than 3.0 millimeters, and preferably in the range of approximately 1.0 and 3.0 millimeters. Minimization of the distance between the negative and positive electrodes of a sensing pair optimizes the sense signal and virtual eliminates outside disturbances caused by various factors, such as a pacing event. It is critical to sense electrical pulses coming directly from the heart, and minimize the noise (e.g., pacing artifacts).

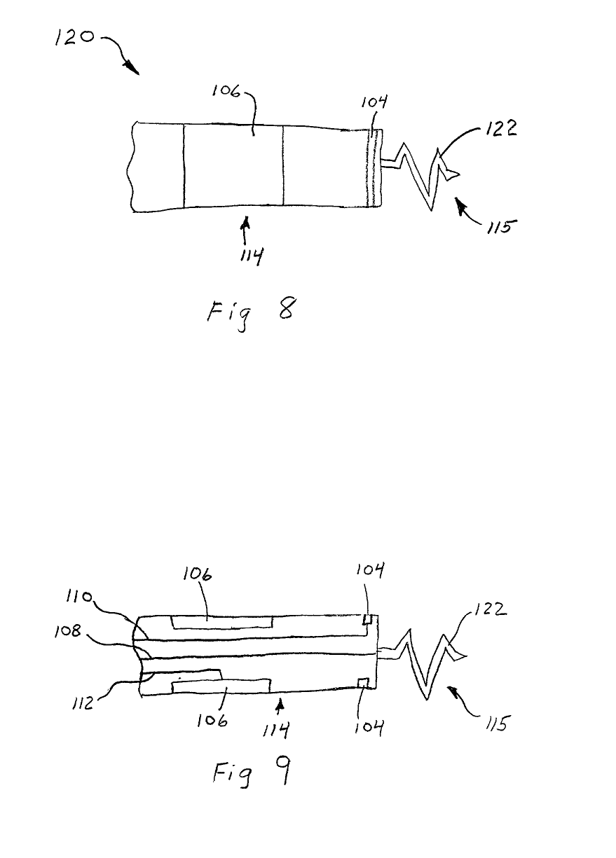

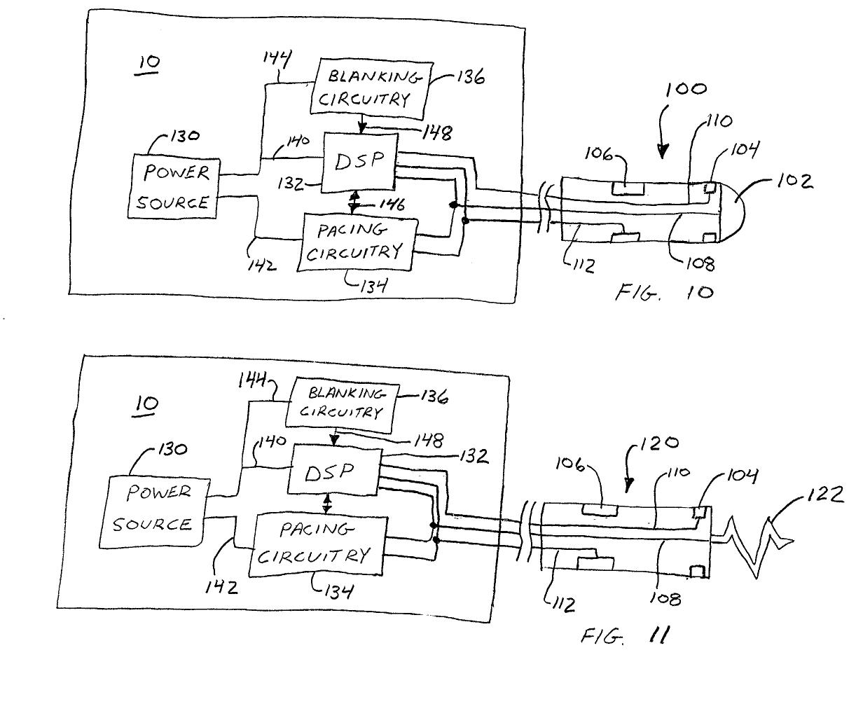

FIGS. 8 and 9 are a side view and a cross-sectional view, respectively, of a second embodiment incorporating the present invention. As shown in FIGS. 8 and 9, tri-polar pacing and sensing lead 120 includes several similar elements and characteristics with tri-polar pacing and sensing lead 100. These similar characteristics and elements have been labeled with identical reference numerals.

Tri-polar pacing and sensing lead 120 includes electrodes 104, 106, and 122, with associated conductors 110, 112, and 124, and body portion 114. Similar to lead 110, lead 120 may be substituted for pacing and sensing leads 16 and 18 shown and described with reference to FIGS. 1-3 and for leads 1 and 7 shown and described with reference to FIG. 4.

In one preferred embodiment, electrode 122 is considered a tip electrode and is located at proximal end 115 of lead 100. Electrode 122 is formed from either a porous material or from any other conducting material, such as platinum. As shown in FIGS. 8 and 9, electrode 122 is fabricated in a “screw-like” configuration such that it can protrude into desired tissue of the heart. The functional aspects of tri-polar pacing and sensing lead 20 are similar to those of tri-polar pacing and sensing lead 100, shown and described with reference to FIGS. 6 and 7. Specifically, tri-polar pacing and sensing lead 120 is capable of providing both local and large area sensing via electrode pairs 104, 122, and 106, 122, respectively. In addition, electrode pair 106, 122 is capable of providing pacing functions as previously discussed.

FIGS. 10 and 11 are schematic diagrams of implantable medical device 10 in accordance with the present invention. All elements of IMD 10 shown in FIGS. 10 and 11 are identical, other than tip electrodes 102 and 122. The specific differences between tip electrodes 102 and 122 have been previously discussed. As shown in FIGS. 10 and 11, IMD 10 includes power source 130, digital signal processing 132, pacing circuitry 134, and blanking circuitry 136.

As shown in FIGS. 10 and 11, conductor 108 electrically couples tip electrode 102 or 122 to both digital signal processing 132 and pacing circuitry 134. Conductor 110 electrically couples ring electrode 104 to digital signal processor 132. Conductor 112 electrically couples ring electrode 106 to both digital signal processor 132 and pacing circuitry 134. Power source 130 is electrically coupled to digital signal processor 132, pacing circuitry 134, and blanking circuitry 136 via electrical connectors 140, 142, and 144, respectively. Digital signal processor 132 is electrically coupled to pacing circuitry 134 via electrical connector 146, while blanking circuitry 136 is electrically coupled to digital signal processor 132 via electrical connector 148.

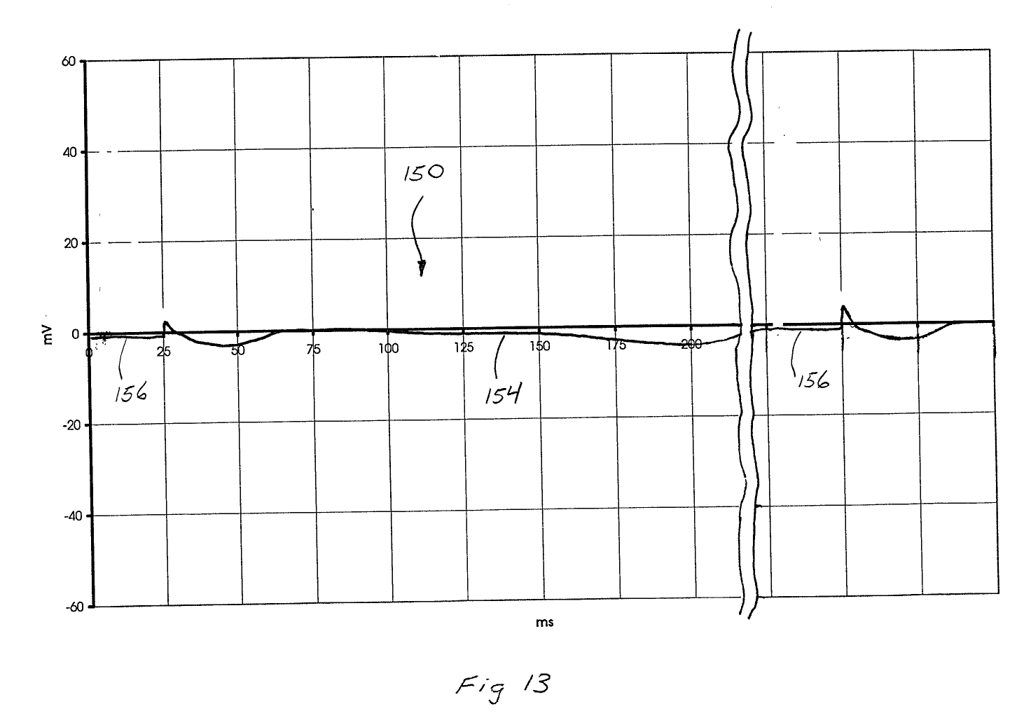

Power source 130, digital signal processor 132, pacing circuitry 134, and blanking circuitry 136 have been previously been described with reference to FIGS. 1-5. However, to summarize, power source 130 provides the necessary power to digital signal processor 132, pacing circuitry 134, and blanking circuitry 136 to properly operate all components and sub-components within each circuit. Digital signal processor 132 receives information and signals relating to heart 8 and specific tissue of heart 8 transmitted from pacing and sensing lead 100. Digital signal processor 132 provides analyzing functions on the received information and signals and produces various output information, such as the graphs shown in FIGS. 12-14. Pacing circuitry 134 provides a pacing electrical signal to heart 8 in order to properly pace heart 8. In one preferred embodiment, pacing circuitry 134 is preprogrammed by a physician in response to a specific condition of heart 8. In another embodiment, pacing circuitry 134 may be changed or altered to provide varying pacing schemes to heart 8 automatically in response to the signals being sensed. This changing pacing signal system may be referred to as a rate response system, which will later be described in greater detail. Blanking circuitry 136 provides a blanking function in which a portion of the sensed signal is blanked or not recorded. The blanking time usually corresponds to the time at which a pacing event occurs and the time immediately thereafter. In one preferred embodiment, the blanking feature lasts in the range of approximately 0-30 milliseconds.

FIGS. 12-14 are various graphs illustrating an electrical signal sensed from heart 8 via tri-polar pacing and sensing lead 100 in accordance with the present invention. The graph shown in FIG. 12 illustrates an unmodified or raw signal that may be sensed from heart 8 via electrodes 102 and 104 of lead 100 or via electrodes 122 and 104 of lead 120. As shown in FIG. 12, signal 150 includes portion 152, which corresponds to a pacing event, and portion 154, which corresponds to a time period between pacing events. At portion 152, IMD 10 is sensing a pacing event as previously described. Thus, a large spike in the pacing signal is recorded. Eventually, the electrically signal corresponding to the pacing event subsides and portion 154 of signal 150 is recorded, corresponding to relatively minimal electrical activity within heart 8 immediately surrounding lead 100. The pattern of heart 8 then repeats.

Under ideal circumstances, IMD 10 would sense and record the entire electrical signal from heart 8. However, in reality, this information may be confusing to a physician reviewing the graphical information. In particular, signal 150 often includes various electrical pulses and signals which indicate specific conditions or attributes of heart 8 which are critical to a physician determining proper IMD settings. For example, signal 150 may include information relating to muscular tissue of heart 8, a heart abnormality, a tachycardia condition, an arrhythmic condition, or blood flow within heart 8. A physician may confuse a sensed pacing artifact with an indication of one of the above referenced conditions or attributes of heart 8.

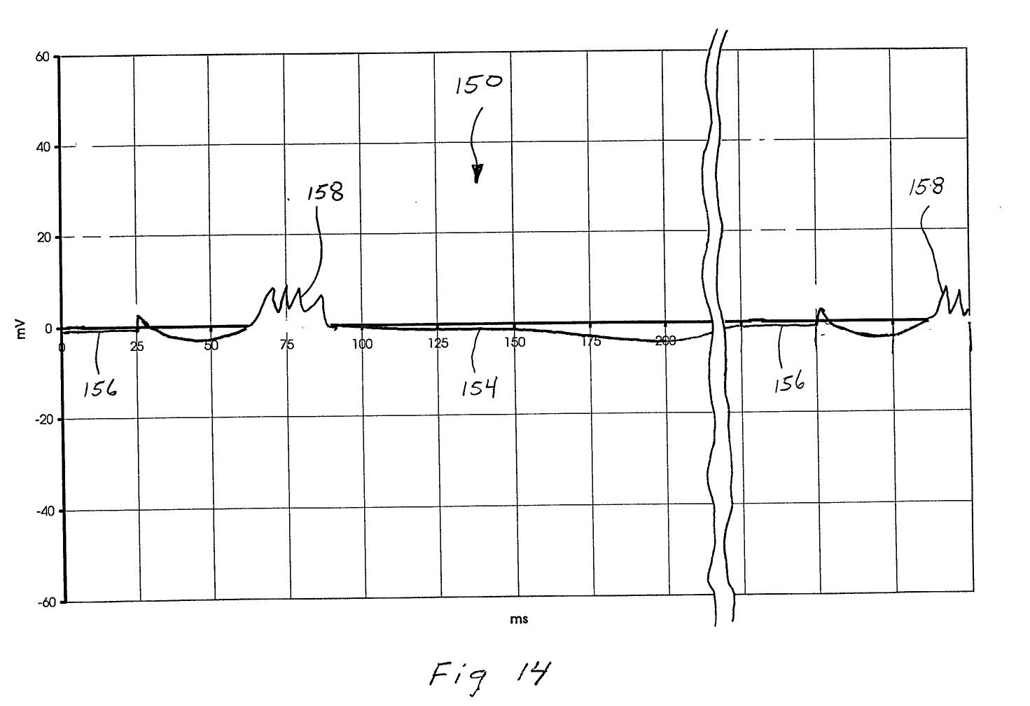

In order to prevent confusion or misdiagnosis of a pacing event as a condition or attribute of heart 8, blanking circuitry 136, shown in FIGS. 10 and 11, blanks out the portion of the sensed signal. As shown in FIG. 13, portion 152 of signal has been blanked and rather than a pronounced spike in signal 150, flat line 156 is shown. It is known in the art that a flat line, such as flat line 156, represents a pacing event. In one preferred embodiment, signal 150 is blanked in the range of approximately 0-30 milliseconds. Prior art IMDs required a significantly greater blanking time, due to spacing between the electrodes. For example, in prior art IMDs, a blanking period in the range of approximately 50-200 milliseconds would be required.

A minimal blanking time period is desirous. Any electrical pulses or signals representative of real events of heart 8 during a blanking time period are not included in a corresponding graph. Therefore, it is critical to minimize the blanking time period. Leads 100 and 120 provide a tool to minimize the blanking time frame due to the proximal location of electrodes 102 or 122 to electrode 104. Electrodes 102, 122 and 104 provide local sensing of electrical signals or pulses from a portion of heart 8. It is understood by those in the art that the precision of sensing has an inverted correlation to the distance between electrodes.

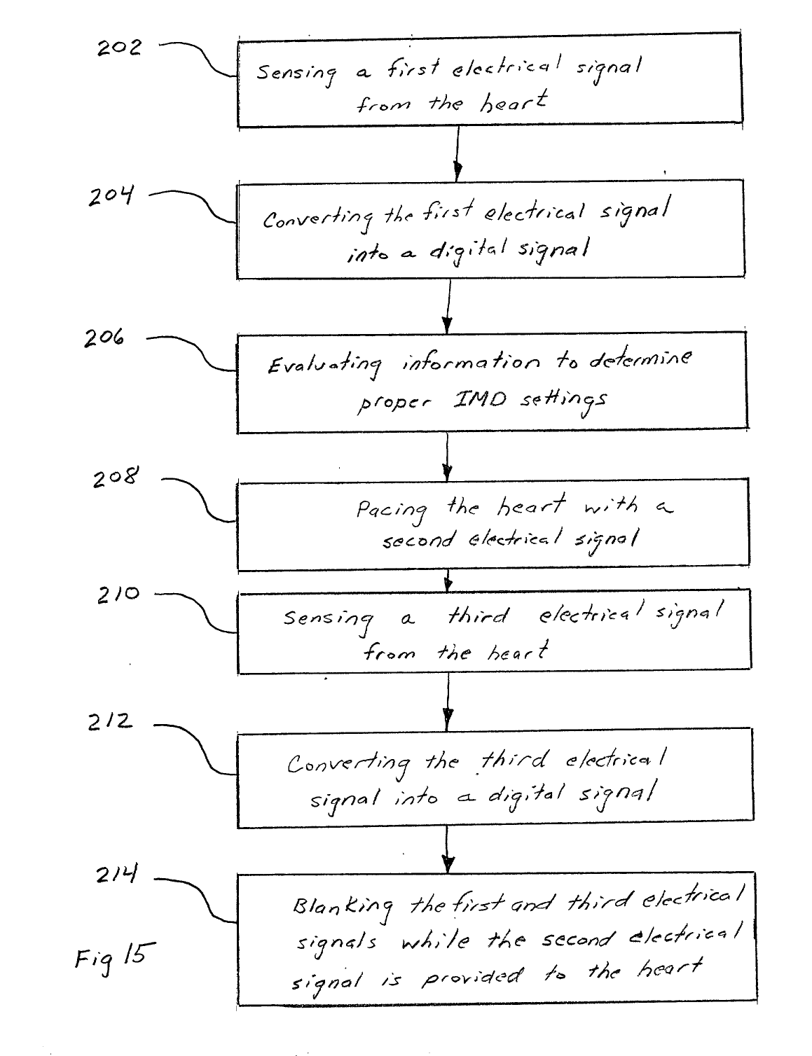

FIG. 14 illustrates signal 150 that includes both blanking section 156 and pulse 158. Pulse 158 represents a real electrical pulse sensed from heart 8 or a portion thereof. Signal 150, shown in FIG. 14, represents a signal which may be evaluated by a physician to order to determine proper IMD settings to optimize the functionality of IMD 10. Alternatively, in a rate response system, IMD 10 may automatically adjust specific settings in response to signal 150, thereby providing an optimal pacing signal to heart 8.

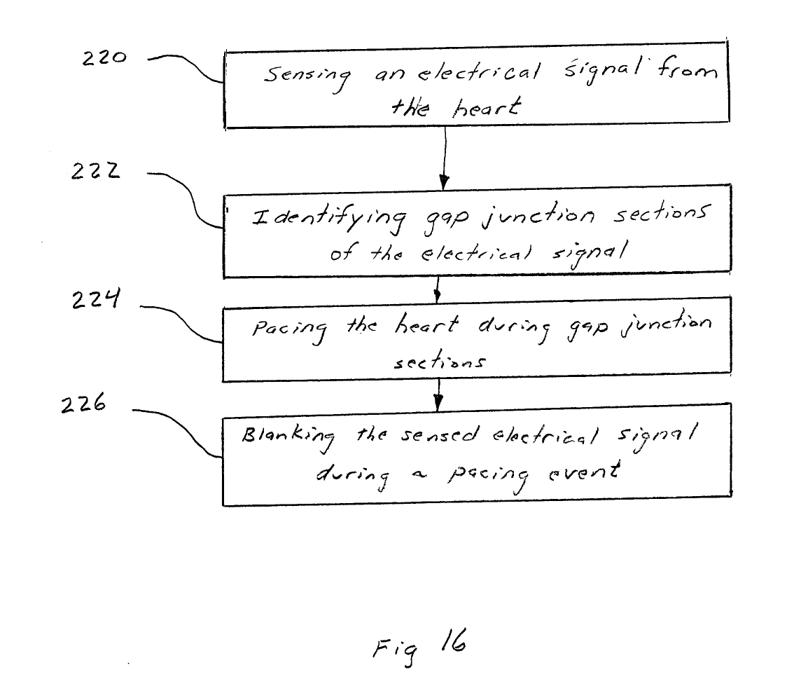

FIG. 15 is a flow chart illustrating various steps in the pacing and sensing of heart 8 in accordance with the present invention. At step 202, a first electrical signal or series of electrical pulses (sensing signal) from heart 8 is sensed between tip electrode 102 or 122 and ring electrode 104. As shown in FIGS. 6-11, tip electrodes 102 and 122 are positioned at distal end 115 of lead 100, while ring electrode 104 is positioned on lead 100 proximal from tip electrodes 102 or 122. In one embodiment, the first electrical signal or series of electrical pulses may include various information, such as information relating to muscular tissue of the heart, a heart abnormality, a tachycardia condition, an arrhythmic condition, or blood flow within heart 8, for example. At step 204, the first electrical signal or series of electrical pulses (sensing signal) is converted into a digital signal by digital signal processor 132. At step 206, information from digital signal processor 132 relating to heart 8 is evaluated, such as by a physician, to determine proper IMD settings. At step 208, a second electrical signal or series of electrical pulses (pacing signal) is provided to heart 8 via tip electrode 102 or 122 and ring electrode 106. At step 210, a third electrical signal or series of electrical pulses (sensing signal) is sensed between tip electrode 102 or 122 and ring electrode 106. At step 212, the third electrical signal or series of electrical pulses in converted into a second digital signal by digital signal processor 132. At step 214, the first and third electrical signals or series of electrical pulses (sensing signals) are blanked while the second electrical signal or series of electrical pulses (pacing signal) are provided to heart 8.

FIG. 16 is a flow chart illustrating various steps in another embodiment for the pacing and sensing of heart 8 in accordance with the present invention. At step 220, a sensed electrical signal or series of electrical pulses is sensed from heart 8 between tip electrode 102 and 122 and ring electrode 104. The sensed electrical signal may include a variety of information relating to heart 8, as previously discussed with reference to FIG. 15. At step 222, gap junction sections of the sensed electrical signal is identified, indication times at which there is minimal heart activity. As is known in the art, the optimal time to provide a pacing signal to heart 8 is during these gap junction sections. At step 224, a pacing electrical signal or series of electrical pulses is provided to heart 8 between tip electrode 102 or 122 and ring electrode 106 during the identified gap junctions. In one embodiment, a physician modifies the settings of IMD 10 to provide pacing during gap junction sections. In another embodiment, IMD 10 automatically modifies its settings to provide pacing during gap junction sections. Thus, IMD 10 is constantly evaluating sensing events and providing optimum pacing signals or pulses based upon sensed signals. At step 226, a portion of the sensed electrical signal or series of electrical pulses is blanked while the pacing electrical signal or series of electrical pulses is provided to heart 8.

The methods of sensing and pacing shown and described with reference to FIGS. 15 and 16 include only a minute portion of the pacing and sensing of IMD 10. It is understood that the methods of FIGS. 15 and 16 may be constantly repeated throughout the life of IMD10. In other words, information is constantly being gathered and revisions to pacing settings may be adjusted periodically.

The preceding specific embodiments are illustrative of the practice of the invention. It is to be understood, therefore, that other expedients known to those skilled in the art or disclosed herein, may be employed without departing from the invention or the scope of the appended claims. For example, the present invention is not limited to pacing or sensing a time specific period, but rather includes pacing and sensing electrical signals or series of electrical pulses of various time segments, depending upon the situation or configuration. In addition, the specific configuration and layout of the electrical lead used in this disclosure should not be limited by the embodiments in the Figures, except where specific dimensions or configurations are articulated.

In the claims, means-plus-function clauses are intended to cover the structures described herein as performing the recited function and not only structural equivalence but also equivalent structures. Thus, although a nail and a screw may not be structural equivalence in that a nail employs a cylindrical surface to secure wooden parts together, whereas a screw employs a helical surface, an environment of fastening wooden parts a nail and a screw are equivalent structures.

What is claimed is:

1. A tri-polar pacing and sensing lead associated with an implantable medical device, the lead comprising:
a first electrode positioned at a distal end of the lead;
a second electrode positioned proximal from the first electrode, wherein a first set of electrical signals are capable of being sensed from a heart of a patient via the first and second electrodes; and
a third electrode positioned proximal from the second electrode, wherein a second set of electrical signals for pacing the heart are capable of being provided to the heart via the first and second electrodes.

2. The lead of claim 1, wherein the first electrode is a common electrode, common to both the second and third electrodes.

3. The lead of claim 1, wherein the first electrode is a tip electrode.

4. The lead of claim 3, wherein the tip electrode is a metal material configured to engage tissue of a heart of a patient.

5. The lead of claim 3, wherein the tip electrode is formed from porous material.

6. The lead of claim 1, wherein the second electrode is a ring electrode.

7. The lead of claim 1, wherein the third electrode is a ring electrode.

8. The lead of claim 1, wherein the second electrode is positioned in the range of approximately 1.0-3.0 millimeters from the first electrode.

9. The lead of claim 1, wherein the third electrode is positioned in the range of approximately 10.0-30.0 millimeters from the first electrode.

10. A tri-polar pacing and sensing lead associated with an implantable medical device, the lead comprising:
a tip electrode;
a first ring electrode located in the range of approximately 1.0-3.0 millimeters from the tip electrode; and
a second ring electrode located in the range of approximately 10.0 to 30.0 millimeters from the tip electrode.

11. The lead of claim 10, wherein the tip electrode is a common electrode, common to both the first ring electrode and the second ring electrode.

12. The lead of claim 10, wherein the tip electrode is a metal material configured to engage tissue of a heart of a patient.

13. The lead of claim 10, wherein the tip electrode is fabricated from a porous material.

14. An implantable medical device for sensing electrical pulses from a heart of a patient and for pacing the heart, the implantable medical device comprising:
a power source;
a lead capable of insertion into the heart of the patient, the lead further comprising:
a first electrode positioned at a distal end of the lead;
a second electrode positioned proximal from the first electrode, wherein a first set of electrical signals are capable of being sensed from the heart via the first and second electrodes;
a third electrode positioned proximal from the second electrode such that a second set of electrical signals are capable of being provided to the heart via the first and third electrodes;
a digital signal processor electrically coupled to the first and second electrodes capable of converting the first set of electrical signals into a digital signal; and
pacing electrical circuitry electrically coupled to the first and third electrodes and coupled to the digital signal processor, the pacing electrical circuitry capable of providing the second set of electrical signals to the heart via the first and third ring electrodes.

15. The implantable medical device of claim 14, wherein the first electrode of the lead is a common electrode, common to both the second and third electrodes.

16. The implantable medical device of claim 14, wherein the first electrode is a tip electrode.

17. The implantable medical device of claim 16, wherein the first electrode is configured to engage tissues of the heart of the patient.

18. The implantable medical device of claim 16, wherein the first electrode is fabricated from a porous material.

19. The implantable medical device of claim 14, wherein the second electrode is a ring electrode.

20. The implantable medical device of claim 14, wherein the third electrode is a ring electrode.

21. The implantable medical device of claim 14, wherein the second electrode is positioned in the range of approximately 1.0-3.0 millimeters from the first electrode.

22. The implantable medical device of claim 14, wherein the third electrode is positioned in the range of approximately 10.0-30.0 millimeters from the first electrode.

23. The implantable medical device of claim 14, and further comprising:
blanking electrical circuitry electrically coupled to the digital signal processor capable of blanking a portion of the digital signal corresponding to a pacing event.

24. A method of providing pacing electrical pulses to a heart of a patient and of sensing electrical pulses from the heart of the patient, the method comprising:
electrically coupling a tip electrode of a lead and a first ring electrode positioned on the lead proximal from the tip electrode to a digital signal processor;
sensing a first electrical signal from the heart between the tip electrode and the first ring electrode;
converting the first electrical signal into a digital signal;
electrically coupling the digital signal processor to pacing electrical circuitry;
electrically coupling the tip electrode and a second ring electrode positioned on the lead proximal from the first ring electrode to the pacing electrical circuitry; and
transmitting a pacing electrical signal between the tip and second ring electrode.

25. The method of claim 24, wherein the step of sensing the first electrical signal further comprises:
sensing the first electrical signal which includes information relating to muscular tissue of the heart.

26. The method of claim 24, wherein the step of sensing the first electrical signal further comprises:
sensing the first electrical signal which includes information relating to a heart abnormality.

27. The method of claim 24, wherein the step of sensing the first electrical signal further comprises:
sensing the first electrical signal which includes information relating to a tachycardia condition.

28. The method of claim 24, wherein the step of sensing the first electrical signal further comprises:
sensing the first electrical signal which includes information relating to an arrhythmic condition.

29. The method of claim 24, wherein the step of sensing the first electrical signal further comprises:
sensing the first electrical signal which includes information relating to blood flow within the heart.

30. The method of claim 24, wherein the step of transmitting a pacing electrical signal further comprises:
transmitting a current pulse between the tip and second ring electrode to pace the heart.

31. The method of claim 24, wherein the step of transmitting a pacing electrical signal further comprises:
transmitting a pacing electrical signal between the tip and second ring electrode, thereby causing a portion of the heart to contract.

32. The method of claim 24, and further comprising:
sensing a second electrical signal from the heart between the tip electrode and the second ring electrode.

33. The method of claim 24, wherein the step of electrically coupling the tip electrode of the lead and the first ring electrode to the digital signal processor further comprises:
electrically coupling the tip electrode of the lead and the first ring electrode positioned on the lead in the range of approximately 1.0-3.0 millimeters from the tip electrode to the digital signal processor.

34. The method of claim 24, wherein the step of electrically coupling the tip electrode and second ring electrode to the pacing electrical circuitry further comprises:
electrically coupling the tip electrode and the second ring electrode positioned in the range of approximately 10.0-30.0 millimeters from the tip electrode to the pacing electrical circuitry.

35. The method of claim 24, and further comprising:
blanking the first electrical signal while the pacing electrical signal is transmitted to the tip and second ring electrode.

36. A method of sensing electrical pulses from a heart of a patient and of providing pacing electrical pulses to the heart of the patient, the method comprising:
sensing a first electrical signal from the heart between a tip electrode of a lead and a first ring electrode positioned on the lead proximal from the tip electrode;
evaluating the first electrical signal;
providing a second electrical signal to the heart between the tip electrode and a second ring electrode.

37. The method of claim 36, wherein the step of sensing the first electrical signal further comprises:
sensing electrical signals which include information relating to muscle tissue of the heart.

38. The method of claim 36, wherein the step of sensing the first electrical signal further comprises:
sensing electrical signals which include information relating to a heart abnormality.

39. The method of claim 36, wherein the step of sensing the first electrical signal further comprises:
sensing electrical signals which include information relating to a tachycardia condition.

40. The method of claim 36, wherein the step of sensing the first electrical signal further comprises:
sensing electrical signals which include information relating to an arrhythmic condition.

41. The method of claim 36, wherein the step of sensing the first electrical signal further comprises:
sensing electrical signals which include information relating to blood flow within the heart.

42. The method of claim 36, wherein the step of providing the second electrical signal to the heart further comprises:
providing a current pulse to the heart between the tip electrode and the second ring electrode.

43. The method of claim 36, wherein the step of providing the second electrical signal to the heart further comprises:
providing a second electrical signal to the heart between the tip electrode and the second ring electrode, thereby causing a portion of the heart to contract.

44. The method of claim 36, and further comprising:
sensing a third electrical signal between the tip electrode and the second ring electrode.

45. The method of claim 36, wherein the step of sensing the first electrical signal from the heart further comprises:
sensing the first electrical signal from the heart between the tip electrode of the lead and the first ring electrode positioned on the lead in the range of approximately 1.0-3.0 millimeters from the tip electrode.

46. The method of claim 36, wherein the step of providing the second electrical signal to the heart further comprises:
providing the second electrical signal to the heart between the tip electrode and the second ring electrode positioned in the range of approximately 10.0-30.0 millimeters from the tip electrode.

47. The method of claim 36, and further comprising:
blanking the first electrical signal while the second electrical signal is provided to the heart.

48. A method of providing pacing electrical pulses to a heart of a patient and of sensing electrical pulses from the heart of the patient, the method comprising:
sensing a sensed electrical signal from the heart of the patient between a tip electrode and a first ring electrode located on a lead proximal from the tip electrode;
converting the sensed electrical signal into a digital signal;
providing an electrical signal representative of the digital signal to pacing electrical circuitry; and
providing a pacing electrical signal to the heart of the patient between the tip electrode and a second ring electrode located on the lead proximal from the first ring electrode.

49. The method of claim 48, wherein the step of sensing the sensed electrical signal from the heart further comprises:
sensing electrical signals which include information relating to muscle tissue of the heart.

50. The method of claim 48, wherein the step of sensing the sensed electrical signal from the heart further comprises:
sensing electrical signals which include information relating to a heart abnormality.

51. The method of claim 48, wherein the step of sensing the sensed electrical signal from the heart further comprises:
sensing electrical signals which include information relating to a tachycardia condition.

52. The method of claim 48, wherein the step of sensing the sensed electrical signal from the heart further comprises:
sensing electrical signals which include information relating to an arrhythmic condition.

53. The method of claim 48, wherein the step of sensing the sensed electrical signal from the heart further comprises:
sensing electrical signals which include information relating to blood flow within the heart.

54. The method of claim 48, wherein the step of providing the pacing electrical signal to the heart further comprises:
providing a current burst to the heart of the patient between the tip electrode and the second ring electrode.

55. The method of claim 48, wherein the step of providing the pacing electrical signal to the heart further comprises:
providing a pacing electrical signal to the heart of the patient between the tip electrode and the second ring electrode, thereby causing a portion of the heart to contract.

56. The method of claim 48, and further comprising:
sensing a second sensed electrical signal from the heart of the patient between the tip electrode and the second ring electrode.

57. The method of claim 48, wherein the step of sensing the sensed electrical signal from the heart further comprises:
sensing the sense electrical signal from the heart of the patient between the tip electrode and the first ring electrode positioned in the range of approximately 1.0-3.0 millimeters from the tip electrode.

58. The method of claim 48, wherein the step of providing the pacing electrical signal to the heart further comprises:
providing the pacing electrical signal to the heart between the tip electrode and the second ring electrode positioned in the range of approximately 10.0-30.0 millimeters from the tip electrode.

59. The method of claim 48, and further comprising:
blanking the sensed electrical signal while the pacing electrical signal is provided to the heart of the patient.

60. A method of providing pacing electrical pulses to a heart of a patient and of sensing electrical pulses from the heart of the patient, the method comprising:
sensing a sensed electrical signal from the heart between a tip electrode of a lead and a first ring electrode positioned on the lead proximal from the tip electrode;
identifying gap junction sections of the sensed electrical signal indicating times at which there is minimal heart activity; and
providing a pacing electrical signal to the heart during the identified gap junction sections of the sensed electrical signal between the tip electrode and the second ring electrode positioned on the lead proximal from the first ring electrode.

61. The method of claim 60, wherein the step of sensing the sensed electrical signal from the heart further comprises:
sensing electrical signals which include information relating to muscle tissue of the heart.

62. The method of claim 60, wherein the step of sensing the sensed electrical signal from the heart further comprises:
sensing electrical signals which include information relating to a heart abnormality.

63. The method of claim 60, wherein the step of sensing the sensed electrical signal from the heart further comprises:
sensing electrical signals which include information relating to a tachycardia condition.

64. The method of claim 60, wherein the step of sensing the sensed electrical signal from the heart further comprises:
sensing electrical signals which include information relating to an arrhythmic condition.

65. The method of claim 60, wherein the step of sensing the sensed electrical signal from the heart further comprises:
sensing electrical signals which include information relating to blood flow within the heart.

66. The method of claim 60, wherein the step of providing the pacing electrical signal to the heart further comprises:
providing a current burst to the heart of the patient between the tip electrode and the second ring electrode.

67. The method of claim 60, wherein the step of providing the pacing electrical signal to the heart further comprises:
providing the pacing electrical signal to the heart of the patient between the tip electrode and the second ring electrode, thereby causing a portion of the heart to contract.

68. The method of claim 60, and further comprising:
sensing a second sensed electrical signal from the heart of the patient between the tip electrode and the second ring electrode.

69. The method of claim 60, wherein the step of sensing the sensed electrical signal from the heart further comprises:
sensing the sensed electrical signal from the heart of the patient between the tip electrode and the first ring electrode positioned in the range of approximately 10.0-3.0 millimeters from the tip electrode.

70. The method of claim 60, wherein the step of providing the pacing electrical signal to the heart further comprises:
providing the pacing electrical signal to the heart between the tip electrode and the second ring electrode positioned in the range of approximately 10.0-30.0 millimeters from the tip electrode.

71. The method of claim 60, and further comprising:
blanking the sensed electrical signal while the pacing electrical signal is provided to the heart of the patient.

72. An implantable medical device for providing pacing electrical pulses to a heart of a patient and for sensing electrical pulses from the heart of the patient, the implantable medical device comprising:
sensing means for sensing a sensed electrical signal from the heart between a tip electrode of a lead and a first ring electrode positioned on the lead proximal from the tip electrode;
identifying means for identifying gap junction sections of the sensed electrical signal indicating times at which there is minimal heart activity; and
pacing means for providing a pacing electrical signal to the heart during the identified gap junction sections of the sensed electrical signal between the tip electrode and a second ring electrode positioned on the lead proximal from the first ring electrode.

73. An implantable medical device for providing pacing electrical pulses to a heart of a patient and for sensing electrical pulses from the heart of the patient, the implantable medical device comprising:
means for electrically coupling a tip electrode of a lead and a first ring electrode positioned on the lead proximal from the tip electrode to a digital signal processor;
means for sensing a first electrical signal from the heart between the tip electrode and the first ring electrode;
means for converting the first electrical signal into a digital signal;
means for electrically coupling the digital signal processor to pacing electrical circuitry;
means for electrically coupling the tip electrode and a second ring electrode positioned on the lead proximal from the first ring electrode to the pacing electrical circuitry; and
means for transmitting a pacing electrical signal between the tip and second ring electrode.

74. An implantable medical device for providing pacing electrical pulses to a heart of a patient and for sensing electrical pulses from the heart of the patient, the implantable medical device comprising:
means for sensing a first electrical signal from the heart between a tip electrode of a lead and a first ring electrode positioned on the lead proximal from the tip electrode;
means for evaluating the first electrical signal;
means for providing a second electrical signal to the heart between the tip electrode and a second ring electrode.

75. An implantable medical device for providing pacing electrical pulses to a heart of a patient and for sensing electrical pulses from the heart of the patient, the implantable medical device comprising:
means for sensing a sensed electrical signal from the heart of the patient between a tip electrode and a first ring electrode located on a lead proximal from the tip electrode;
means for converting the sensed electrical signal into a digital signal;
means for providing an electrical signal representative of the digital signal to pacing electrical circuitry; and
means for providing a pacing electrical signal to the heart of the patient between the tip electrode and a second ring electrode located on the lead proximal from the first ring electrode.
